(19) United States
(12) Patent Application Publication
(10) Pub. No.: US 2021/0138170 A1
(43) Pub. Date: May 13, 2021

(54) SYSTEM AND METHOD FOR MONITORING A PATIENT DURING OXYGEN THERAPY

(71) Applicant: KONINKLIJKE PHILIPS N.V., EINDHOVEN (NL)

(72) Inventors: Nikolaos KARAMOLEGKOS, Boston, MA (US); Francesco Vicario, Boston, MA (US); Cornelis Conradus Adrianus Maria van Zon, Everett, MA (US); Haibo Wang, Melrose, MA (US)

(21) Appl. No.: 17/022,548

(22) Filed: Sep. 16, 2020

Related U.S. Application Data
(60) Provisional application No. 62/934,089, filed on Nov. 12, 2019.

(51) Int. Cl. A61M 16/00; A61M 16/04

(52) U.S. Cl. CPC A61M 16/0051; A61M 16/0003; A61M 2205/583; A61M 16/0463; A61M 16/024

(57) ABSTRACT

A system configured to monitor a patient undergoing an oxygen therapy includes a housing configured to be connected to a patient interface that delivers the oxygen therapy to the patient; a plurality of sensors disposed in or on the housing and configured to generate output signals conveying information about one or more patient/system interaction attributes associated with the oxygen therapy; and a computer system that comprises one or more physical processors operatively connected with the plurality of sensors, the physical processors being programmed with computer program instructions which, when executed cause the computer system to: determine the one or more patient/system interaction attributes associated with the oxygen therapy based on the information in the output signals; and generate output information for communication to the patient and/or a caregiver based on the output signals.

### CROSS-REFERENCE TO RELATED APPLICATIONS

This patent application claims the priority benefit under 35 U.S.C. § 119(e) of U.S. Provisional Application No. 62/934,089 filed on Nov. 12, 2019, the contents of which are herein incorporated by reference.

### BACKGROUND

### 1. Field

The present patent application discloses a system and a method for monitoring a patient during oxygen therapy, for example, mechanical ventilation or other nasal/oxygen therapies.

### 2. Description of the Related Art

Mechanical ventilation (MV) is typically instituted when a patient is unable to maintain adequate ventilation, and hence gas exchange, on their own. It is estimated that every year nearly 1.5 million patients across the United States require some form of ventilation assist and this number is set to increase. Despite the undoubted benefits of mechanical ventilation, there is room for improvement in connection with effectiveness and patient comfort.

In the case of noninvasive ventilation (NIV), correct placement of the mask (or other patient interface) used to ventilate the patient, as well as correct tightness/looseness for proper therapy delivery and high patient comfort is desired.

In addition, placing the patients in a semi-recumbent position (between 30 and 45 degrees) is often desired for a variety of reasons. However, despite this guideline, compliance remains low and technical solutions have limitations. For instance, the inclinometers that can be built into the hospital bed have been developed. However, they are often unreliable as patients tend to slide down the bed, thus reducing the effectiveness of the inclination.

Therefore, for the above and additional reasons, improved systems and methods are desired.

### SUMMARY

Accordingly, one or more aspects of the present patent application relate to a system configured to monitor a patient undergoing an oxygen therapy. The system comprises: a housing configured to be connected to a patient interface that delivers the oxygen therapy to the patient; a plurality of sensors disposed in or on the housing and configured to generate output signals conveying information about one or more patient/system interaction attributes associated with the oxygen therapy; and a computer system that comprises one or more physical processors operatively connected with the plurality of sensors. The one or more physical processors is programmed with computer program instructions which, when executed cause the computer system to: determine the one or more patient/system interaction attributes associated with the oxygen therapy based on the information in the output signals; and generate output information for communication to the patient and/or a caregiver based on the output signals.

Another aspect of the present patent application relates to a method for monitoring a patient undergoing an oxygen therapy. The method is implemented by a computer system that comprises one or more physical processors executing machine readable instructions that, when executed, perform the method. The method comprises providing a plurality of sensors in or on a housing, the housing configured to be connected to a patient interface that delivers the oxygen therapy to the patient; obtaining, from the plurality of sensors, output signals conveying information about one or more patient/system interaction attributes associated with the oxygen therapy; determining the one or more patient/system interaction attributes associated with the oxygen therapy based on the information in the output signals; and generating output information for communication to the patient and/or a caregiver based on the output signals.

Yet another aspect of the present patent application relates to a system configured to monitor a patient undergoing an oxygen therapy. The system comprises: a housing configured to be connected to a patient interfacing means that delivers the oxygen therapy to the patient; a plurality of sensing means disposed in or on the housing and configured to generate output signals conveying information about one or more patient/system interaction attributes associated with the oxygen therapy; and controlling means operatively connected with the plurality of sensing means. The controlling means are configured to: determine the one or more patient/system interaction attributes associated with the oxygen therapy based on the information in the output signals; and generate output information for communication to the patient and/or a caregiver based on the output signals.

These and other objects, features, and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosure.

### BRIEF DESCRIPTION OF THE DRAWINGS

FIG. 1 shows a prior art capnography sensor system;

FIG. 2 shows a system for monitoring a patient undergoing an oxygen therapy in accordance with an embodiment of the present patent application,

FIG. 3 shows a sensor system of the system for monitoring the patient undergoing the oxygen therapy in accordance with an embodiment of the present patent application, wherein a built-in camera is visualized;

FIGS. 4A and 4B show the system for monitoring the patient undergoing the oxygen therapy in accordance with an embodiment of the present patent application, wherein the system is being used by the patient undergoing an invasive ventilation therapy;

FIG. 5 shows the sensor system of the system for monitoring the patient undergoing the oxygen therapy in accordance with an embodiment of the present patent application, wherein the sensor system includes a plurality of sensors comprising capnograph sensor, camera(s); motion sensor(s)/accelerometers and/or inclination sensor(s), wherein the plurality of sensors comprising camera(s); motion sensor(s)/accelerometers and/or inclination sensor(s) are in the same housing as capnography sensor;

FIG. 6 shows the system for monitoring the patient undergoing the oxygen therapy in accordance with an embodiment of the present patent application, wherein the system is configured to monitor patient comfort via video (and possibly motion) analysis;

FIG. 7 shows the system for monitoring the patient undergoing the oxygen therapy in accordance with an embodiment of the present patent application, wherein the system is configured to detect and alert self-extubation attempts;

FIG. 8 shows the system for monitoring the patient undergoing the oxygen therapy in accordance with an embodiment of the present patent application, wherein the system is configured to monitor the patent inclination;

FIG. 9 shows the sensor system for monitoring the patient undergoing the oxygen therapy in accordance with another embodiment of the present patent application, wherein camera and other sensor modules are shown as an add-on to a standard capnography sensor, wherein the plurality of sensors comprising camera(s); motion sensor(s)/accelerometers and/or inclination sensor(s) are in a different modular housing component than the modular housing component configured to receive a capnography sensor; and

FIG. 10 shows a method for monitoring a patient undergoing an oxygen therapy in accordance with an embodiment of the present patent application.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

FIGS. 2-9 schematically illustrate a system 10 configured to monitor a patient 12 undergoing an oxygen therapy. In some embodiments, system 10 comprising a housing 27 configured to be connected to a patient interface 16 that delivers the oxygen therapy to the patient; a plurality of sensors 18 disposed in or on housing 27 and configured to generate output signals conveying information about one or more patient/system interaction attributes associated with the oxygen therapy; and a computer system 22 that comprises one or more physical processors 24 operatively connected with plurality of sensors 18. In some embodiments, one or more physical processors 24 is programmed with computer program instructions which, when executed cause the computer system to: determine the one or more patient/system interaction attributes associated with the oxygen therapy based on the information in the output signals; and generate output information for communication to the patient and/or a caregiver based on the output signals.

In some embodiments, the output information includes an alert. In some embodiments, the output information is generated in response to determining that the one or more patient/system interaction attributes associated with the oxygen therapy meet an alert condition. In some embodiments, the oxygen therapy to the patient is provided by an oxygen therapy system 14 configured to deliver oxygen to the patient through patient interface 16.

In some embodiments, system 10 includes just plurality of (various) sensors 18, housing 27 on or in which plurality of sensors 18 is carried, and computer system 22. In some embodiments, as described in detail below, patient interface 16 can be conventional, and system 10 can be configured to interface and/or be connected with such patient interfaces. In some embodiments, housing 27 of system 10 is configured to interface and/or be connected with such patient interfaces. In such an embodiment, housing 27 is configured to receive plurality of sensors 18 and computer system 22 therein or thereon.

In some embodiments, housing 27 includes a connector/airway adapter 30 (as shown in FIGS. 4 and 11) configured to connect housing 27 and patient interface 16. In some embodiments, housing 27 is connected to the airway adapter 30 that is placed between endotracheal tube 25 and patient/breathing circuit 17.

In some embodiments, as shown in FIG. 9, housing 27 includes two or more modular housing components 27a and 27b. In some embodiments, one 27a of the two or more modular housing components 27a and 27b is configured to receive a capnography sensor 102. In some embodiments, the other 27b of the two or more modular housing components 27a and 27b is configured to receive plurality of sensors 18 including camera(s) 104; motion sensor/accelerometer 106 and/or inclination sensor 108. In some embodiments, the number of modular housing components may vary. In some embodiments, modular housing components 27a and 27b of housing 27 are configured to detachably attached to each other using any mechanism as would be appreciated by one skilled in the art. In some embodiments, capnography sensor 102 is optional and, in such an embodiment, housing 27 and its modular housing components 27a and 27b are configured to receive plurality of sensors 18 including camera(s) 104; motion sensor/accelerometer 106 and/or inclination sensor 108.

In some embodiments, housing 27 is configured to be easily attached to oxygen therapy system 14 including patient interface 16 using any mechanism as would be appreciated by one skilled in the art. In some embodiments, housing 27 may have any shape and configuration and includes housing body in which electronic components/circuits can be arranged. In some embodiments, housing 27 is configured to receive plurality of sensors 18 and computer system 22. In some embodiments, housing 27 is configured to also accommodate a battery for the power supply, and a sensor for detecting location data.

In some embodiments, plurality of sensors 18 is configured to disposed at a predetermined vicinity of the patient 12. In some embodiments, plurality of sensors 18 is configured to disposed on the patient interface at a distance of between 3 and 10 inches from the patient. In some embodiments, plurality of sensors 18 is disposed at a distance of between 3 and 10 inches from patient's face to determine discomfort of patient 12. In some embodiments, plurality of sensors 18 is disposed at a distance of between 3 and 10 inches from patient's upper body so as to determine inclination of patient 12.

In some embodiments, plurality of sensors 18 includes one or more cameras 104 configured to detect the discomfort of patient 12 and/or provide evaluation of the placement of patient interface 16 on patient 12 both for the efficiency of the oxygen therapy and the comfort of patient 12; and one or more motion sensors 106 configured to detect any sudden movement of endotracheal tube 25 of patient interface 16. In some embodiments, plurality of sensors 18 includes one or more inclination sensors 108 configured to detect an inclination of patient 12.

In some embodiments, plurality of sensors (or sensing means) 18 includes at least two sensors. In some embodiments, the at least two of plurality of sensors 18 are selected from the group consisting of one or more cameras 104, one or more motion sensors 106, and one or more inclination sensors 108.

In some embodiments, in this patent application, the patient may be interchangeably referred to as a consumer, a user, an individual or a subject. In some embodiments, hardware processors may be interchangeably referred to as physical processors. In some embodiments, machine readable instructions may be interchangeably referred to as computer program instructions. In some embodiments, the ventilator-associated pneumonia may be referred to as VAP. In some embodiments, the noninvasive ventilation may be referred to as NIV.

In some embodiments, the present patent application provides an integrated system 13 for patient monitoring during mechanical ventilation. In some embodiments, system 10 of the present patent application is configured to monitor patient discomfort, attempts of self-extubation, compliance with the ventilation-associated pneumonia bundles, and, in case of noninvasive ventilation, also monitor placement of mask 23. In some embodiments, as shown in FIGS. 3 and 5, the present patent application provides an integrated capnography sensor 102 with a camera 104, a motion sensor 106, and an inclination sensor 108 for patient monitoring during mechanical ventilation.

In some embodiments, camera-based algorithms 112 are configured to assess and monitor patient discomfort. In some embodiments, the motion sensor 106 includes an accelerometer 106. In some embodiments, the motion sensor 106 includes a magnetometer 106. In some embodiments, the motion sensor 106 is configured to detect attempts of self-extubation, whereas the inclinometer/inclination sensor 108 is configured to monitor the inclination of patient 12. Monitoring patient inclination is used for reducing the risk of the ventilation-associated pneumonia (VAP). In some embodiments, in the case of the noninvasive ventilation, system 10 of the present patent application is also configured to provide feedback on mask 23 or patient interface 16 placement.

In some embodiments, system 10 is configured to detect, reduce, and/or prevent adverse patient/system interaction events that are associated with ventilation therapy, including patient discomfort and related complications (e.g., self-extubation), and the ventilation-associated pneumonia. In some embodiments, system 10 is configured to be embedded in equipment already routinely used in the Intensive Care Unit (ICU), without the burden of having to set up and maintain additional devices or requiring additional space in an already crowded environment. In some embodiments, system 10 includes an integrated capnography sensor (capnograph) 102 with a built-in camera (or a system of cameras) 104 along with motion sensor(s) (e.g., accelerometer or magnetometer) 106 and inclination sensor(s) (e.g., inclinometer) 108 that aims to address the above needs.

In some embodiments, oxygen system or oxygen therapy system or oxygen supply 14 may also be referred to as a mechanical ventilation system. In some embodiments, oxygen system 14 may be an invasive mechanical ventilation system. In some embodiments, oxygen system 14 may be a non-invasive mechanical ventilation system.

In some embodiments, oxygen system 14 includes an oxygen source 19 and a ventilator 11. In some embodiments, ventilator 11 is operatively connected to oxygen source 19, and ventilator 11 is configured to deliver a mixture of oxygen and ambient air to the patient through a breathing circuit 17. In some embodiments, ventilator 11 is operatively connected to oxygen source 19 to receive a supply of oxygen therefrom. In some embodiments, ventilator 11 is configured to mix the received flow of oxygen with ambient air drawn by ventilator 11 and deliver the mixed oxygen and air to patient 12 through breathing circuit 17. In some embodiments, ventilator 11 may also be referred to as oxygen blender 11. In some embodiments, oxygen system 14 may also include air pump 15 configured to deliver other nasal therapies. In some embodiments, in the invasive mechanical ventilation system, breathing circuit 17 includes an endotracheal (or a tracheostomy) tube 25.

In some embodiments, oxygen therapy may include a mechanical ventilation therapy. In some embodiments, oxygen therapy may include a nasal therapy. In some embodiments, the oxygen therapy may include a non-invasive mechanical ventilation therapy. In some embodiments, mask 23 is used to deliver oxygen to patient 12 during the invasive mechanical ventilation therapy. In some embodiments, mask 23 is part of patient interface 16.

In some embodiments, the oxygen therapy may include an invasive mechanical ventilation therapy. In some embodiments, endotracheal tube 25 is used to deliver oxygen to patient 12 during the invasive mechanical ventilation therapy. In some embodiments, endotracheal tube 25 is part of patient interface 16. In some embodiments, endotracheal tube 25 may also be referred to as tracheostomy tube. In some embodiments, the oxygen therapy may include other nasal therapies.

In some embodiments, patient interface 16 is operatively coupled to the patient/delivery circuit to communicate the oxygen-enriched breathing gas to the nasal cavity/airway of patient 12. In some embodiments, delivery circuit may include a conduit and/or patient interface 16. Delivery circuit may sometimes be referred to as patient interface 16. In some embodiments, the conduit may include a flexible length of hose, or other conduit, either in single-limb or dual-limb configuration that places patient interface 16 in fluid communication with oxygen system 14. In some embodiments, the conduit forms a flow/fluid path through which the flow of oxygen-enriched breathing gas is communicated between patient interface 16 and oxygen system 14. In some embodiments, delivery circuit may also be referred to as breathing circuit 17. In some embodiments, patient interface 16 includes mask 23 in case of the non-invasive mechanical ventilation therapy. In some embodiments, patient interface 16 includes endotracheal tube 25 in case of the invasive mechanical ventilation therapy. In some embodiments, patient interface 16 may be configured to deliver oxygen-enriched breathing gas to the nasal cavity/airway of patient 12. As such, patient interface 16 may include any appliance/device suitable for this function. In some embodiments, patient interface 16 is configured to be removably coupled with another interface being used to deliver oxygen therapy to patient 12. For example, patient interface 16 may be configured to engage with and/or be inserted into other interface appliances/devices. In some embodiments, patient interface 16 may be configured to engage the airway/the nasal cavity of patient 12 without an intervening device. In some embodiments, patient interface 16 may include one or more of an a nasal cannula, nasal interface, nasal prongs, nasal pillows, a nasal mask, a nasal/oral mask, a full-face mask, a total facemask, and/or other interface devices that communicate a flow of oxygen-enriched breathing gas with an airway/a nasal cavity of patient 12. The present patent application is not limited to these examples, and contemplates delivery of the oxygen-enriched breathing gas to patient 16 using any subject interface.

In some embodiments, oxygen system/supply 14 includes an oxygen blender 11, an air pump 15, and an oxygen source 19. In some embodiments, oxygen supply 14 includes oxygen blender 11 and oxygen source 19. In some embodiments, oxygen source 19 is configured to supply the oxygen to oxygen blender 11. In some embodiments, oxygen source 19 is an oxygen tank or an oxygen cylinder, which stores compressed, oxygen enriched gas.

In some embodiments, in some nasal therapies, air pump 15 is configured to control flow rate of the oxygen-enriched breathing gas being delivered to the nasal cavity of patient 12. In some embodiments, air pump 15 is a variable speed pump or a variable speed blower. In some embodiments, air pump 15 may include valves, stepper motor, flow rate sensors, and drive electronics. In some embodiments, air pump 15 is configured to control flow rate of the oxygen-enriched breathing gas being delivered to the nasal cavity of patient 12 as determined by output signals/commands received from computer system 22. In some embodiments, air pump 15 is configured to control flow rate of the oxygen-enriched breathing gas being delivered to the nasal cavity of patient 12 based on the patient's input. In some embodiments, air pump 15 is used in nasal therapy systems and is not used in mechanical ventilation systems (e.g., invasive or non-invasive systems).

In some embodiments, ventilator or oxygen blender 11 is interchangeably referred to as an oxygen/air blender. In some embodiments, oxygen blender 11 is configured to control Fio₂ level of the oxygen-enriched breathing gas being delivered to the nasal cavity of patient 12. In some embodiments, oxygen blender 11 is configured to mix/blend oxygen from oxygen source 19 and ambient air to the desired concentration as determined by output signals/commands received from computer system 22. In some embodiments, oxygen blender 11 is configured to mix/blend oxygen from oxygen source 19 and ambient air to the desired concentration based on the patient's input. In some embodiments, oxygen blender 11 may include valves, stepper motor, and drive electronics.

In some embodiments, system 10 comprises an user interface 21 configured to enable patient 12 to select a predetermined flow parameters of the oxygen-enriched breathing gas being delivered to the nasal cavity of patient 12.

In some embodiments, user interface 21 may be configured to provide an interface between system 10 and patient 12 through which patient 12 can provide information to and receive information from system 10. This enables data, results, and/or instructions and any other communicable items, collectively referred to as “information,” to be communicated between patient 12 and system 10. Examples of interface devices suitable for inclusion in user interface 21 include a keypad, buttons, switches, a keyboard, knobs, levers, a display screen, a touch screen, speakers, a microphone, an indicator light, an audible alarm, and a printer. Information may be provided to patient 12 by user interface 21 in the form of auditory signals, visual signals, tactile signals, and/or other sensory signals. It is to be understood that other communication techniques, either hard-wired or wireless, are also contemplated herein as the user interface. For example, in one embodiment, user interface 21 may be integrated with a removable storage interface provided by electronic storage 132. In this example, information is loaded into system 10 from removable storage (e.g., a smart card, a flash drive, a removable disk, etc.) that enables the user(s) to customize system 10. Other exemplary input devices and techniques adapted for use with system 10 as user interface include, but are not limited to, an RS-232 port, RF link, an IR link, modem (telephone, cable, Ethernet, internet or other). In short, any technique for communicating information with system 10 is contemplated as the user interface.

In some embodiments, system 10 configured to monitor the patient 12 undergoing an invasive mechanical ventilation therapy includes capnography sensor 102, camera(s) 104, motion sensor(s) 106, inclination sensor(s) 108, and/or other sensor(s).

In some embodiments, system 10 configured to monitor the patient 12 undergoing a non-invasive mechanical ventilation therapy includes camera(s) 104, motion sensor(s) 106, inclination sensor(s) 108, and/or other sensor(s). In some embodiments, such a system may optionally include capnography sensor 102.

In some embodiments, system 10 configured to monitor the patient 12 undergoing a nasal therapy includes camera(s) 104, motion sensor(s) 106, inclination sensor(s) 108, and/or other sensor(s). In some embodiments, such a system may include capnography sensor 102.

In some embodiments, plurality of sensors 18 is configured to generate output signals conveying information about one or more patient/system interaction attributes associated with the oxygen therapy.

In some embodiments, the one or more patient/system interaction attributes associated with the oxygen therapy comprises an interaction attribute (i.e., patient interaction attribute) for discomfort of the patient undergoing the oxygen therapy, an interaction attribute (i.e., system interaction attribute) associated with an attempt of self-extubation by the patient undergoing the oxygen therapy via invasive mechanical ventilation, an interaction attribute (i.e., system interaction attribute) for placement of the patient interface on the patient both for efficiency of the oxygen therapy and comfort of the patient undergoing the oxygen therapy, and/or an interaction attribute (i.e., patient interaction attribute, for example, inclination of patient 12) associated with a ventilator associated pneumonia.

In some embodiments, plurality of sensors 18 includes one or more cameras 104 configured to detect the discomfort of the patient and/or provide evaluation of the placement of the patient interface on patient 12 both for the efficiency of the oxygen therapy and the comfort of patient 12; one or more motion sensors 106 configured to detect any sudden movement of an endotracheal tube 25 of patient interface 26; and/or one or more inclination sensors 108 configured to detect an inclination of patient 12.

In some embodiments, plurality of sensors 18 is configured to generate output signals conveying information related to patient comfort during the oxygen therapy, patient inclination during the oxygen therapy, and/or self-extubation during invasive mechanical ventilation therapy. As another example, the information may be obtained from one or more monitoring devices (e.g., patient comfort monitoring device, self-extubation monitoring device, patient inclination monitoring device, or other patient/system interaction monitoring devices). In some embodiments, one or more patient/system interaction monitoring devices and associated sensors 18 may be configured to monitor patient comfort during the oxygen therapy. In some embodiments, one or more patient/system interaction monitoring devices and associated sensors 18 may be configured to monitor self-extubation during invasive ventilation therapy. In some embodiments, system is configured to provide an indication of inclination that is recommended for VAP prevention. In some embodiments, one or more patient/system interaction monitoring devices and associated sensors 18 may be configured to monitor patient inclination during the oxygen therapy. These patient/system interaction monitoring devices may include plurality of sensors 18, such as camera(s) 104, motion sensor(s) 106, inclination sensor(s) 108, or other sensors.

In some embodiments, each of plurality of sensors 18 includes a transmitter for sending signals and a receiver for receiving the signals. In some embodiments, plurality of sensors 18 is configured to communicate wirelessly with computer system 22. As shown in FIG. 2, in some embodiments, one or more sensor(s) 18 are configured to be operatively connected with computer system 22 and/or one or more physical processors 24 of computer system 22. In some embodiments, plurality of sensors 18 is in communication with a database 132.

In some embodiments, the information related to one or more patient/system interaction attributes associated with the oxygen therapy may be obtained from the database 132 that is being updated in real-time by plurality of sensors 18.

In one scenario, a monitoring device may obtain information (e.g., based on information from plurality of sensors 18), and provide information to computer system 22 (e.g., comprising server 24) over a network (e.g., network 150) for processing. In another scenario, upon obtaining the information, the monitoring device may process the obtained information, and provide processed information to computer system 22 over a network (e.g., network 150). In yet another scenario, the monitoring device may automatically provide information (e.g., obtained or processed) to computer system 22 (e.g., comprising server 24). In some embodiments, sensors 18 may be placed close to the patient and/or at the system's other locations, with appropriate compensation algorithms to estimate the corresponding patient/system interaction attributes in proximity of patient 12. In some embodiments, server 24 includes one or more physical/hardware processors 24. In FIG. 2, database 132 is shown as a separate entity, but, in some embodiments, database 132 could be part of computer system 22.

In some embodiments, system 10 includes one or more hardware processors 24 operatively connected with plurality of sensors 18. As shown in FIG. 2, system 10 may comprise server 24 (or multiple servers 24). In some embodiments, server 24 comprises oxygen therapy associated attributes determination subsystem 112, monitor subsystem 114, alert subsystem 116 or other components or subsystems.

As will be clear from the discussions above and below, in some embodiments, system 10 includes computer system 22 that has one or more physical/hardware processors 24 programmed with computer program/machine readable instructions that, when executed cause computer system 22 to obtain information or data from plurality of sensors 18.

In some embodiments, capnography sensor 102 is configured for measuring a level of carbon dioxide in exhaled breath of patient 12. The measurement of CO₂ in exhaled breath is generally known as capnography. If patient 12 is on a mechanical ventilator/respirator, the CO₂ continues along a respiratory pathway to the respirator. En route or at the respirator, the level of CO₂ is measured.

In some embodiments, capnography sensor 102 is configured for measuring a level of carbon dioxide in exhaled breath of patient 12 during mechanical ventilation therapies (e.g., invasive or non-invasive mechanical ventilation therapies). In some embodiments, capnography sensor 102 is not used during some nasal therapies.

In some embodiments, camera(s) 104 with associated computer vision algorithms 122 (as shown in FIGS. 6 and 7) are configured to analyze facial expressions (e.g., pain, fear, and/or other expressions and emotions) and provide indicators of patient discomfort.

In some embodiments, camera(s) 104 with associated computer vision algorithms 122 may together be referred to as a camera system.

In some embodiments, camera(s) 104 are configured to monitor facial expressions/images of patient 12 in order to detect patient discomfort. In some embodiments, camera(s) 104 configured to be positioned such that the camera(s) have a generally unobstructed view of the patient's face. In some embodiments, camera(s) 104 are configured to may be monitor facial expressions/images of patient 12 at predetermined intervals. In some embodiments, camera(s) 104 is configured to monitor facial expressions/images of patient 12 upon receiving a signal from other sensors of system 10.

In some embodiments, in the noninvasive ventilation therapy, camera(s) 104 with associated computer vision algorithms 122 are configured to analyze the area around facial mask of patient 12 and detect high-pressure points in order to provide a quantitative evaluation of facial mask placement in terms of patient discomfort (e.g., tight seal of mask 23) and therapy efficiency (e.g., leaks due to a loose seal of mask 23).

In some embodiments, camera(s) 104 is configured to provide and/or analyze any movement associated with patient 12 and/or a location and position of patient 12. In some embodiments, analysis of the movement associated with patient 12 and/or the location and position of patient 12 may be used to detect patient discomfort.

In some embodiments, system 10 includes a single camera. In some embodiments, system 10 includes two or more cameras. In some embodiments, each camera may be positioned differently from the other cameras, and each camera may serve a unique purpose. For example, one camera may monitor patient discomfort and the other camera may monitor the placement of mask 23. In some embodiments, the number of cameras may vary and may depend on different views of patient 12 and their surrounding environment desired. In some embodiments, system 10 is also configured to monitor the surrounding environment of the patient with the additional cameras.

In some embodiments, cameras may have same type of lenses. In some embodiments, cameras may have different type of lenses. In some embodiments, the type of camera lenses may include an ultra-wide-angle lens for wider field of view of patient 12 and their surrounding environment. In some embodiments, one or more camera(s) include RGB camera, 3D camera, depth camera, infrared camera, etc.

Discomfort is often linked to the ventilator systems in both invasive and noninvasive ventilation. For example, endotracheal tube 25 that is used in the invasive ventilation systems may be uncomfortable to patients who breathe spontaneously. In addition, general discomfort, whether caused by intubation, other conditions, or routine ICU interventions, can make patient 12 prone to self-extubation. During the noninvasive ventilation, a good mask seal is crucial; large air leaks interfere with the effectiveness of the noninvasive ventilation therapy/treatment, while small air leaks may irritate patient 12, causing conjunctivitis or creating noise. Tight seal is, however, undesirable because it can cause discomfort and mask-related complications, such as facial skin erythema, skin breakdown, rash, conjunctivitis or dryness of the mucosa.

In some embodiments, as shown in FIG. 6, camera(s) 104 and accelerometer(s) 106 are configured to detect discomfort of patient 12. In some embodiments, as shown in FIG. 6, camera(s) 104 and accelerometer(s) 106 are configured to assist in proper placement and fit of mask 23 in non-invasive mechanical ventilation therapy systems.

In some embodiments, gyroscope (and/or inclinometer) 108 is configured to assist in proper placement and fit of the cannula in nasal therapy systems.

In some embodiments, other type of sensors may be configured to detect discomfort of patient 12. For example, audio sensors may be configured to detect discomfort of patient 12. For example, audio sensors may be used along with camera(s) 104 to detect discomfort of patient 12. In some embodiments, other type of sensors may determine that patient 12 is experiencing discomfort by comparing their respective sensor outputs with corresponding baseline data.

In some embodiments, in the case of the noninvasive ventilation (NIV) therapy, correct placement of the mask 23 (or other patient interface 16) used to ventilate patient 12 is important. For example, when the mask 23 (or other patient interface 16) is too tight, the mask becomes uncomfortable for patient 12 and the high contact pressure is a known cause of skin damage. When the mask 23 (or other patient interface 16) is too loose, the mask 23 offers passageways for the pressurized air in breathing circuit 17 to escape to the ambient, interfering with the ventilator operation and jeopardizing the delivery of the prescribed non-invasive ventilation therapy.

In some embodiments, one or more camera(s) 104 with associated computer vision algorithms 122 are configured to analyze the area around facial mask of patient 12 (e.g., in a noninvasive mechanical ventilation therapy) and detect high-pressure points in order to provide a quantitative evaluation of facial mask placement in terms of patient discomfort (i.e., to provide tight seal) and therapy (e.g., noninvasive mechanical ventilation therapy) efficiency (i.e., to prevent leaks due to a loose seal).

In some embodiments, in addition, as shown in FIG. 7, built-in sensor array 105 (e.g., including camera(s) 104 and motion sensor/accelerometer 106) is configured to record movement of breathing tube 25 and detect any attempt of patient self-extubation.

In some embodiments, one or more motion sensor(s) 106 are configured to record movement of breathing tube 25 and detect any attempt of patient self-extubation. In some embodiments, one or more motion sensor(s) 106 are configured to identify movements of breathing tube 25 and detect any attempt of patient self-extubation or other unplanned extubation. In some embodiments, camera(s) are configured to provide visual information about the patient trying to pull endotracheal tube 25 out. It has been shown that some of these unplanned extubations may require re-intubation.

In some embodiments, as shown in FIG. 8, one or more inclinometer sensor(s) 108 are configured to provide real-time feedback of the patient's inclination in order to increase compliance to the ventilation-associated pneumonia prevention guidelines (e.g., ventilation-associated pneumonia (VAP) bundles). It has been found that elevating the patient head position (e.g., maintain the patient's head/upper body raised between 30 and 45 degrees) is a provision included in the so-called “VAP bundles” used in hospitals to reduce the incidence of the ventilator-associated pneumonia.

It is also generally known that ventilator-associated pneumonia occurs due to the passage of bacteria through the endotracheal (or tracheostomy) tube 25 (e.g., placed in the patient's trachea, or through a hole in the front of the patient's neck) of breathing circuit 17. In some embodiments, inclinometer sensor 108 is configured to provide real-time feedback of the patient's head/upper body elevation in a semi-recumbent position. This prevents the source of infection from getting into the lung by reduction in gastroesophageal reflux and can potentially reduce the ventilator-associated pneumonia.

FIG. 3 shows an integrated sensor system of system 10 in accordance with an embodiment of the present patent application, wherein a built-in camera 104 is visualized.

An existing, commercially available, capnography sensor 102′ shown in FIG. 1. This prior art sensor 102′ is compared with sensor system 13 (capnography sensor 102 with integrated plurality of sensors 18 included therein) of the present patent application (as shown in FIG. 3). Although the embodiment in FIG. 3 shows a single camera, two or more cameras (with same or different type of lenses, like an ultra-wide-angle lens for wider field of view) can also be considered if different views of patient 12 and their surrounding environment are desired.

FIGS. 4A and 4B show the sensor system 13 of system 10 in accordance with an embodiment of the present patent application, wherein the sensor system is being used by patient 12 undergoing an invasive ventilation therapy.

As shown in FIG. 4A and FIG. 4B as an exemplary case of an invasively ventilated patient, the capnography sensor 102 is typically connected to breathing circuit 17, proximally to patient 12, via a connector/airway adapter 30. In the case of the noninvasive ventilator, an unobstructed view of patient 12 and of the facial mask/patient interface 23/17 is guaranteed.

FIG. 5 shows sensor system 13 of system 10 in accordance with an embodiment of the present patent application, wherein sensor system 13 includes plurality of sensors comprising camera(s) 104; motion sensor(s) 106 and/or inclination sensor(s) 108.

FIG. 5 shows a schematic diagram of the proposed integrated sensor system 13 and its internal components: 1) capnograph (existing sensor) 102, 2) camera module 104 and 112, 3) accelerometer 106 (or magnetometer; not shown), and/or 4) inclinometer 108.

In some embodiments, plurality of sensors 18 comprising camera(s) 104; motion sensor(s)/accelerometers 106 and/or inclination sensor(s) 108 are in the same housing 27 as capnography sensor 102.

FIG. 6 shows system 10 in accordance with an embodiment of the present patent application, wherein system 10 is configured to monitor and/or alert patient comfort via video (and possibly motion) analysis.

FIGS. 6-8 show possible embodiments of the proposed integrated sensor system 13 for different applications. Such embodiments can be considered as applications of the entire integrated sensor or as separate components that can work independently from the capnography sensor 102.

In particular, FIG. 6 displays a block diagram of the steps for assessing patient comfort by analyzing video from the camera module 104 and 112. Analysis can also be augmented with motion from the embedded motion sensor (accelerometer in this embodiment) 106. Computer vision capabilities may be enabled via, for example, deep trained neural networks. Training of a neural network could be done by collecting large amounts of videos of facial expressions of incubated patients and manually categorizing them into two classes: comfort and pain. Once trained, by feeding a short video, the networks will output a possibility of the patient not being in comfort; this can be used as indicator for monitoring or alerting systems. To equip capnography sensor 102 with this computer vision ability, specified vision processing chips may be embedded to support onboard edge computing.

FIG. 7 shows sensor system 13 in accordance with an embodiment of the present patent application, wherein sensor system 13 is configured to alert self-extubation attempts. Detection of attempts from the patient to self-extubated by pulling endotracheal tube 25 out may be assessed by analyzing motion via the embedded motion sensor (accelerometer 106 in some embodiments) as shown in the block diagram of FIG. 7. Video feed from camera 104 also be incorporated in the detection algorithm.

FIG. 8 shows the system in accordance with an embodiment of the present patent application, wherein the system is configured to monitor the patent inclination. FIG. 8 shows the embodiment for monitoring the patient's inclination via the embedded inclination sensor.

FIG. 9 shows sensor system 13 in accordance with another embodiment of the present patent application, wherein camera module 104 and 112 and sensor modules (i.e., motion sensor(s)/accelerometer 106 and/or inclination sensor 108) are shown as an add-on to the standard capnography sensor 102. In some embodiments, plurality of sensors 18 comprising camera(s) 104; motion sensor(s)/accelerometers 106 and/or inclination sensor(s) 108 are in a different modular housing component 27b than the modular housing component 27a configured to receive a capnography sensor 102.

In some embodiments, camera(s) 104, motion sensor(s) 106 and inclination sensor(s) 108 are independent of the capnography sensor 102, either as a separate accessory or as an add-on on the capnography sensor 102. In some embodiments, camera(s) 104 and sensor modules 106 and 108 are shown as an add-on to the standard/existing capnography sensor 102.

In some embodiments, determination subsystem 112 is configured to determine the one or more patient/system interaction attributes associated with the oxygen therapy based on the information in the output signals from plurality of sensors 18. In some embodiments, determination subsystem 112 includes video and motion analysis algorithms 122.

In some embodiments, monitor subsystem 114 is configured to continuously or intermittently track the determined one or more patient/system interaction attributes associated with the oxygen therapy (including, but not limited to, patient's discomfort, patient's inclination, patient's movement, movement of patient's interface, etc.) while patient 12 is being ventilated in a home or clinical setting.

In some embodiments, monitor subsystem 114 is configured to determine that patient 12 is experiencing discomfort by comparing their respective patient/system interaction attributes (patient's movement, facial expressions, audio signals) with corresponding baseline data. In some embodiments, monitor subsystem 114 is configured to determine that patient 12 may have higher risk of VAP by comparing their respective patient/system interaction attributes (patient's inclination) with corresponding baseline data. In some embodiments, monitor subsystem 114 is configured to determine that patient 12 may attempt a self-extubation by comparing their respective patient/system interaction attributes (patient's movement, facial expressions, audio signals, movement of tubes of patient interface) with corresponding baseline data. In some embodiments, monitor subsystem 114 is configured to determine proper placement of patient interface 16 on patient 12 both for efficiency of the oxygen therapy and comfort of patient 12 undergoing the oxygen therapy by comparing their respective patient/system interaction attributes (patient's movement, facial expressions, audio signals, movement of masks of patient interface) with corresponding baseline data.

In some embodiments, for example, a determination is made as to whether the determined one or more patient/system interaction attributes are greater than a threshold value. If it is determined that the threshold has not been met, then the patient/system interaction attributes are continually measured/determined. If, however, it is determined that the threshold has been met, then an alert is generated. Persons of ordinary skill in the art will recognize that the threshold being “met” may correspond to a particular measured characteristic value exceeding the threshold, exceeding the threshold by a particular amount, equaling the threshold, being less than the threshold, or being less than the threshold by a particular amount, and may depend on the particular characteristic being measured/determined.

In some embodiments, when one or more patient/system interaction attributes associated with the oxygen therapy (including, but not limited to, patient's discomfort, patient's inclination, patient's movement, movement of patient's interface, etc.) while patient 12 is being ventilated in a home or clinical setting exceeds the range and rules that are prescribed, then a designed protocol is configured to be invoked that may issue an alert/notification to a monitoring clinical provider or team requiring an action to acknowledge and respond.

In some embodiments, alert subsystem 116 is configured to generate an alert for communication to patient 12 and/or a caregiver of patient 12 in response to determining that the one or more patient/system interaction attributes associated with the oxygen therapy meet an alert condition.

In some embodiments, the alert generated may correspond to audio data, text data, image data, or any other form of data capable of alerting one or more alert systems. For example, the alert may correspond to an audible tone to be output by alert system, which may correspond to a mobile device including a speaker that alerts a caregiver to check on the status of patient 12. In one embodiment, the alert corresponds a text message, telephone call, email, or any other type of digital message to be rendered by a user device of a caregiver and/or clinician.

In some embodiments, alert subsystem 116 is configured to alert the patient's care team about the changes in these patient/system interaction attributes via an automated informational message intended to be an early warning and can be sent through system 10.

In some embodiments, the methods within system 10 use advanced statistical analytics, as well as, machine learning and artificial intelligence to determine advance early warning messages.

In some embodiments, the alert requires the clinical team/caregiver to intervene with patient 12, requires the clinical team/caregiver to change the settings of patient inclination, requires patient 12 to change the settings of patient interface/mask or requires the clinical team/caregiver to change the settings of patient interface/endotracheal tube. In some embodiments, alternatively, system 10 is configured to automatically adjust its settings to resolve the alert condition. In some embodiments, the alert requires the clinical team to reevaluate if patient 12 is on the right therapy or medical device to consider alternative or better treatment options.

In some embodiments, system 10 is configured to issue an alert message derived from the alert subsystem 116. In some embodiments, the informational alert generated can be displayed locally on system 10, stored on system 10, or transmitted remotely to a medical information system.

In some embodiments, computer system 22 is configured to notify a clinician of the determined change in one or more patient/system interaction attributes associated with oxygen therapy. In some embodiments, when the change indicates a patient needs medical attention within the specified time period, computer system 22 is configured to generate audio and/or visuals alerts and/or messages notifying clinicians thereof. It is contemplated that such a message can be provided to the clinicians via communication network 150. In some embodiments, computer system 22 is also configured to notify only (and all) medical specialists needed for the case. In some embodiments, the alert requires the clinical team to intervene with patient 12, requires the clinical team/caregiver to change the settings of patient inclination, requires patient 12 to change the settings of patient interface/mask or requires the clinical team/caregiver to change the settings of patient interface/endotracheal tube. In some embodiments, alternatively, system 10 can automatically adjust its settings to resolve the alert condition.

In some embodiments, system 10 is configured to be used with nasal cannula systems. In some embodiments, in such a system, motion sensors 106 may be used in the cannula of the nasal cannula systems for activity tracking or fall detection for elderly population/users, etc. (e.g., for home applications during oxygen therapy). In some embodiments, gyroscope 106 (and/or inclinometer 108) may also be used in the nasal cannula systems to assist in proper placement and fit of the cannula. In some embodiments, camera 104 may also be incorporated in the nasal cannula systems for activity tracking or fall detection for elderly population/users, etc. (e.g., for home applications during oxygen therapy).

In some embodiments, system 10 may be designed and used independently of the CO₂ sensor, either for intubated ventilated patients or for non-invasively ventilated patients.

In some embodiments, the non-invasively ventilated systems do not always have the CO₂ sensor.

Referring to FIG. 10, a method 200 for monitoring patient 12 undergoing an oxygen therapy is provided. The oxygen therapy to patient 12 is provided by oxygen therapy system 14 configured to deliver oxygen to patient 12 through patient interface 16. In some embodiments, method 200 is implemented by computer system 22 that comprises one or more physical processors 24 executing machine readable instructions that, when executed, perform method 200. In some embodiments, method 200 comprises providing plurality of sensors 18 in or on housing 27 at procedure 202. In some embodiments, housing 27 is operatively connected with oxygen therapy system 14 including patient interface 16. In some embodiments, method 200 also includes obtaining, from plurality of sensors 18, output signals conveying information about one or more patient/system interaction attributes associated with the oxygen therapy at procedure 204; and determining the one or more patient/system interaction attributes associated with the oxygen therapy based on the information in the output signals at procedure 206; and generating output information for communication to patient 12 and/or a caregiver based on the output signals.

In some embodiments, the output information includes an alert. In some embodiments, the output information is generated in response to determining that the one or more patient/system interaction attributes associated with the oxygen therapy meet an alert condition. In some embodiments, the oxygen therapy to the patient is provided by an oxygen therapy system 14 configured to deliver oxygen to the patient through patient interface 16.

In some embodiments, system 10 configured to monitor patient 12 undergoing an oxygen therapy is provided. In some embodiments, the oxygen therapy to patient 12 is provided by oxygen therapy delivering means 14 configured to deliver oxygen to patient 12 through patient interfacing means 16. In some embodiments, system 10 comprises housing 27 configured to be connected to patient interfacing means 16 that delivers the oxygen therapy to the patient; a plurality of sensing means 18 disposed in or on housing 27 and configured to generate output signals conveying information about one or more patient/system interaction attributes associated with the oxygen therapy; and controlling means 22, 24 operatively connected with plurality of sensing means 18. In some embodiments, controlling means 22, 24 are configured to: determine the one or more patient/system interaction attributes associated with the oxygen therapy based on the information in the output signals; and generate an alert for communication to patient 12 and/or a caregiver of patient 12 in response to determining that the one or more patient/system interaction attributes associated with the oxygen therapy meet an alert condition.

In some embodiments, the one or more patient/system interaction attributes associated with the oxygen therapy is selected from the group consisting of an interaction attribute for discomfort of patient 12 undergoing the oxygen therapy, an interaction attribute associated with an attempt of self-extubation by patient 12 undergoing the oxygen therapy, an interaction attribute for placement of patient interface 16 on patient 12 both for efficiency of the oxygen therapy and comfort of patient 12 undergoing the oxygen therapy, and an interaction attribute associated with a ventilator associated pneumonia. In some embodiments, plurality of sensing means 18 includes one or more cameras 104 configured to detect the discomfort of patient 12 and/or provide evaluation of the placement of patient interface 16 on patient 12 both for the efficiency of the oxygen therapy and the comfort of patient 12; one or more motion sensors 106 configured to detect any sudden movement of endotracheal tube 25 of patient interface 16; and/or one or more inclination sensors 108 configured to detect an inclination of patient 12. In some embodiments, housing 27 includes two or more modular housing components 27a and 27b, and one 27a of the two or more modular housing components 27a and 27b is configured to receive capnography sensing means 102. In some embodiments, plurality of sensing means 18 is configured to disposed at a predetermined vicinity of patient 12.

In some embodiments, the various computers and subsystems illustrated in FIGS. 2-9 may comprise one or more computing devices that are programmed to perform the functions described herein. The computing devices may include one or more electronic storages (e.g., database 132, or other electronic storages), one or more physical processors programmed with one or more computer program instructions, and/or other components. The computing devices may include communication lines or ports to enable the exchange of information with a network (e.g., network 150) or other computing platforms via wired or wireless techniques (e.g., Ethernet, fiber optics, coaxial cable, WiFi, Bluetooth, near field communication, or other communication technologies). The computing devices may include a plurality of hardware, software, and/or firmware components operating together to provide the functionality attributed herein to the servers. For example, the computing devices may be implemented by a cloud of computing platforms operating together as the computing devices.

The electronic storages may comprise non-transitory storage media that electronically stores information. The electronic storage media of the electronic storages may include one or both of system storage that is provided integrally (e.g., substantially non-removable) with the servers or removable storage that is removably connectable to the servers via, for example, a port (e.g., a USB port, a firewire port, etc.) or a drive (e.g., a disk drive, etc.). The electronic storages may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. The electronic storages may include one or more virtual storage resources (e.g., cloud storage, a virtual private network, and/or other virtual storage resources). The electronic storages may store software algorithms, information determined by the processors, information received from the servers, information received from client computing platforms, or other information that enables the servers to function as described herein.

The processors may be programmed to provide information processing capabilities in the servers. As such, the processors may include one or more of a digital processor, an analog processor, or a digital circuit designed to process information, an analog circuit designed to process information, a state machine, and/or other mechanisms for electronically processing information. In some embodiments, the processors may include a plurality of processing units. These processing units may be physically located within the same device, or the processors may represent processing functionality of a plurality of devices operating in coordination. The processors may be programmed to execute computer program instructions to perform functions described herein of subsystems 112-116 or other subsystems. The processors may be programmed to execute computer program instructions by software; hardware; firmware; some combination of software, hardware, or firmware; and/or other mechanisms for configuring processing capabilities on the processors.

It should be appreciated that the description of the functionality provided by the different subsystems 112-116 described herein is for illustrative purposes, and is not intended to be limiting, as any of subsystems 112-116 may provide more or less functionality than is described. For example, one or more of subsystems 112-116 may be eliminated, and some or all of its functionality may be provided by other ones of subsystems 112-116. As another example, additional subsystems may be programmed to perform some or all of the functionality attributed herein to one of subsystems 112-116.

It should be appreciated that the different subsystems 112-116 performing the operations illustrated in FIG. 2 may reside in system 10 itself. In other embodiments, the different subsystems 112-116 performing the operations illustrated in FIG. 2 may reside in an independent monitoring device.

In some embodiments, system 10 may be used in home healthcare solutions or systems. In some embodiments, system 10 may be used in home respiratory/oxygen systems. In some embodiments, system 10 may be used for mild to moderate chronic obstructive pulmonary disease (COPD) patients. In some embodiments, system 10 may be used for obstructive sleep apnea (OSA) patients. The systems and methods of the present patent application are used in Home ventilation business and/or critical care ventilation business.

In some embodiments, system 10 may also include a communication interface that is configured to send the determined control signals to alert patient or their caregiver through an appropriate wireless communication method (e.g., Wi-Fi, Bluetooth, internet, etc.) or send to other systems for further processing. In some embodiments, system 100 may include a recursive tuning subsystem that is configured to recursively tune its intelligent decision making subsystem using available data or information to provide better overall determination of one or more patient/system interaction attributes associated with the oxygen therapy. In some embodiments, intelligent decision making subsystem, communication interface and recursive tuning subsystem may be part of computer system 22 (comprising server 24). Current capnography sensors only monitor carbon dioxide. The present patent application, however, describes a system with an embedded camera 104 and sensor modules (e.g., accelerometer 106 and/or inclinometer 108) that offer additional monitoring capabilities, like detection and monitoring of patient discomfort, self-extubation, and/or patient inclination.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination. Although the description provided above provides detail for the purpose of illustration based on what is currently considered to be the most practical and preferred embodiments, it is to be understood that such detail is solely for that purpose and that the disclosure is not limited to the expressly disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

What is claimed is:

1. A system configured to monitor a patient undergoing an oxygen therapy, the system comprising:
a housing, the housing configured to be connected to a patient interface that delivers the oxygen therapy to the patient; a plurality of sensors disposed in or on the housing and configured to generate output signals conveying information about one or more patient/system interaction attributes associated with the oxygen therapy; and
a computer system that comprises one or more physical processors operatively connected with the plurality of sensors, the one or more physical processors being programmed with computer program instructions which, when executed cause the computer system to:
determine the one or more patient/system interaction attributes associated with the oxygen therapy based on the information in the output signals; and
generate output information for communication to the patient and/or a caregiver based on the output signals.

2. The system of claim 1, wherein the one or more patient/system interaction attributes associated with the oxygen therapy is selected from the group consisting of an interaction attribute for discomfort of the patient undergoing the oxygen therapy, an interaction attribute associated with an attempt of self-extubation by the patient undergoing the oxygen therapy, an interaction attribute for placement of the patient interface on the patient both for efficiency of the oxygen therapy and comfort of the patient undergoing the oxygen therapy, and an interaction attribute associated with a ventilator associated pneumonia.

3. The system of claim 2, wherein the plurality of sensors includes
one or more cameras configured to detect the discomfort of the patient and/or provide evaluation of the placement of the patient interface on the patient both for the efficiency of the oxygen therapy and the comfort of the patient; and
one or more motion sensors configured to detect any sudden movement of an endotracheal tube of the patient interface.

4. The system of claim 2, wherein the plurality of sensors includes one or more inclination sensors configured to detect an inclination of the patient.

5. The system of claim 1, wherein the housing includes two or more modular housing components, and wherein one of the two or more modular housing components is configured to receive a capnography sensor.

6. The system of claim 1, wherein the plurality of sensors is configured to disposed on the patient interface at a distance of between 3 and 10 inches from the patient.

7. The system of claim 1, wherein the housing includes a connector configured to connect the housing and the patient interface, and where the connector is placed between the patient interface and a patient circuit.

8. The system of claim 1, wherein the output information includes an alert and the output information is generated in response to determining that the one or more patient/system interaction attributes associated with the oxygen therapy meet an alert condition.

9. A method for monitoring a patient undergoing an oxygen therapy, the method being implemented by a computer system that comprises one or more physical processors executing machine readable instructions that, when executed, perform the method, the method comprising:
providing a plurality of sensors in or on a housing, the housing configured to be connected to a patient interface that delivers the oxygen therapy to the patient;
obtaining, from the plurality of sensors, output signals conveying information about one or more patient/system interaction attributes associated with the oxygen therapy;
determining the one or more patient/system interaction attributes associated with the oxygen therapy based on the information in the output signals; and
generating output information for communication to the patient and/or a caregiver based on the output signals.

10. The method of claim 9, wherein the oxygen therapy includes an invasive ventilation therapy, and
wherein the one or more patient/system interaction attributes associated with the oxygen therapy is selected from the group consisting of an interaction attribute for discomfort of the patient undergoing the oxygen therapy, an interaction attribute associated with an attempt of self-extubation by the patient undergoing the invasive ventilation therapy, an interaction attribute for placement of the patient interface on the patient both for efficiency of the oxygen therapy and comfort of the patient undergoing the oxygen therapy, and an interaction attribute associated with a ventilator associated pneumonia.

11. A system configured to monitor a patient undergoing an oxygen therapy, the system comprising:
means configured to be connected to a patient interfacing means that delivers the oxygen therapy to the patient;
a plurality of sensing means disposed in or on the housing and configured to generate output signals conveying information about one or more patient/system interaction attributes associated with the oxygen therapy;
controlling means operatively connected with the plurality of sensing means, wherein the controlling means are configured to:
determine the one or more patient/system interaction attributes associated with the oxygen therapy based on the information in the output signals; and
generate output information for communication to the patient and/or a caregiver based on the output signals.

12. The system of claim 11, wherein the oxygen therapy includes an invasive ventilation therapy, and
wherein the one or more patient/system interaction attributes associated with the oxygen therapy is selected from the group consisting of an interaction attribute for discomfort of the patient undergoing the oxygen therapy, an interaction attribute associated with an attempt of self-extubation by the patient undergoing the invasive ventilation therapy, an interaction attribute for placement of the patient interface on the patient both for efficiency of the oxygen therapy and comfort of the patient undergoing the oxygen therapy, and an interaction attribute associated with a ventilator associated pneumonia.

13. The system of claim 12, wherein the plurality of sensing means includes at least two sensing means, wherein the at least two of the plurality of sensing means are selected from the group consisting of
one or more cameras configured to detect the discomfort of the patient and/or provide evaluation of the placement of the patient interface on the patient both for the efficiency of the oxygen therapy and the comfort of the patient;
one or more motion sensors configured to detect any sudden movement of an endotracheal tube of the patient interface; and
one or more inclination sensors configured to detect an inclination of the patient.

14. The system of claim 11, wherein the housing includes two or more modular housing components, and wherein one of the two or more modular housing components is configured to receive a capnography sensing means.

15. The system of claim 11, wherein the plurality of sensing means is configured to disposed on the patient interface at a distance of between 3 and 10 inches from the patient.
